(19) United States
(12) Patent Application Publication
(10) Pub. No.: US 2022/0390377 A1
(43) Pub. Date: Dec. 8, 2022

(54) TECHNIQUE FOR QUANTITATIVELY DETECTING ALKALINE PHOSPHATASE ACTIVITY IN SEAWATER BASED ON SURFACE-ENHANCED RAMAN SPECTROSCOPY

(71) Applicant: SHANGHAI OCEAN UNIVERSITY, SHANGHAI (CN)

(72) Inventors: JIASONG FANG, SHANGHAI (CN); YONG WU, SHANGHAI (CN); HONGGE ZHANG, SHANGHAI (CN); YULI WEI, SHANGHAI (CN); JUNWEI CAO, SHANGHAI (CN); SICONG LI, SHANGHAI (CN)

(21) Appl. No.: 17/828,607

(22) Filed: May 31, 2022

(30) Foreign Application Priority Data
Jun. 2, 2021 (CN) 202110616047.0

(51) Int. Cl. G01N 21/65

(52) U.S. Cl. CPC G01N 21/658

(57) ABSTRACT

The present disclosure provides a technique for quantitatively detecting alkaline phosphatase (ALP) activities in seawater and other aquatic environments, based on surface-enhanced Raman spectroscopy by taking 5-bromo-4-chloro-3-indolyl phosphate (BCIP) as a substrate and dimethyl sulfoxide (DMSO) as an internal standard. Results show that ALP activity has a good linear correlation with the intensity ratio of a characteristic Raman peak to that of the internal standard peak (600 cm⁻¹/677 cm⁻¹) (R²=0.977). The technique was successfully applied to detect ALP activity of a seawater sample. By extension this technique can also be used in detecting the activity of other microbial extracellular enzymes (e.g., aminopeptidase) in seawater and thus, lays a solid scientific foundation for in-situ detection of the activities of other extracellular enzymes in seawater and other aquatic environments.

### TECHNICAL FIELD

The present disclosure relates to a technique for quantitatively detecting alkaline phosphatase and its activities in seawater based on surface-enhanced Raman spectroscopy, and belongs to the technical field of detection by Raman spectroscopy.

### BACKGROUND

Alkaline phosphatase (ALP) is widely distributed in the marine environment, and can participate in hydrolytic reactions of transforming phosphate compounds from animals, plants, and microorganisms, into micromolecular monomers. The generated micromolecular compounds are transported into microbial cells and provide an energy source for life activities of microorganisms. A plurality of literature data show that bacteria and algae live by nutrients generated by hydrolysis from ALP. The synthesis of ALP by marine microorganisms is affected by the surrounding water environmental conditions. When the dissolved inorganic phosphorus (DIP) is insufficient in the surrounding water, the microorganisms can utilize the inorganic phosphorus within the cells to maintain life activities; when the concentration of the inorganic phosphorus is increased in the surrounding water environment, the microbial cells may absorb and store the inorganic phosphorus; However, when the concentration of inorganic phosphorus is low both in the surrounding water environment and inside the cells, ALP is increasingly expressed by microorganisms and ALP activity is gradually increased to maintain the balance of the inorganic phosphate in the microbial cells. Therefore, ALP activity in the water body reflects the status of nutrient phosphorus and the structure of microbial communities in the marine environment. Research on ALP activity has important significance for uncovering mechanisms of marine microorganisms in driving marine phosphorus cycle. Furthermore, microbial extracellular ALP is involved in marine phosphorus cycle as well as marine carbon cycle. Currently, although the activity of ALP can be detected by the traditional fluorescence method, electrochemical analytical method and the like, these methods are limited by complex and time-consuming sample pretreatment. Therefore, it is urgently needed to develop a rapid and efficient ALP detection method with high sensitivity.

Laser Raman spectroscopy is based on the inelastic scattering caused by energy exchange between laser photons and a molecule of a substance after light irradiates the surface of the substance and can reflect the internal energy level structure of the molecule of the substance to represent molecular vibration. Surface-enhanced Raman spectroscopy (SERS) utilizes an optical enhancement effect of metal nanoparticles such as gold and silver to enhance the Raman spectrum signal of a target molecule adsorbed on the particles, thereby realizing rapid detection of low-concentration substances. In recent years, the method has advantages of rapidness, high sensitivity, no loss, no contact and the like, and is widely used in the fields of food safety, biological detection and the like.

### SUMMARY

In order to overcome the above problems, the present disclosure provides a technique for quantitatively detecting alkaline phosphatase activity in seawater based on surface-enhanced Raman spectroscopy.

A technique for quantitatively detecting alkaline phosphatase in seawater based on surface-enhanced Raman spectroscopy includes the following steps:

a. obtaining a variety of alkaline phosphatase samples with different activities in advance, mixing and incubating the alkaline phosphatase samples with a BCIP solution for a period of time, adding a DMSO solution as a standard solution;

b. dropping a plurality of the standard solutions with different activities onto a surface-enhanced Raman scattering substrate separately, conducting an SERS detection separately, and drawing a standard curve of relations between intensity ratios of obtained SERS signals of the standard solutions with different activities to obtained SERS signal of the DMSO and a logarithm value of the activities of the standard solutions;

c. dropping a solution of a sample to be tested containing DMSO onto a surface-enhanced Raman scattering substrate to directly detect SERS signals of the sample to be tested and the DMSO; and

d. comparing the SERS signals obtained in step c with the standard curve to obtain the activity of the sample to be tested.

Furthermore, in step b and step c, the SERS signal of the DMSO is obtained by selecting a peak intensity of the DMSO at a Raman shift of 677 cm⁻¹.

Furthermore, in step b and step c, the SERS signal of the alkaline phosphatase is obtained by selecting a peak intensity of the alkaline phosphatase at a Raman shift of 600 cm⁻¹.

Furthermore, in step b, the standard curve has a fitted equation of y=0.454*x+0.513 with a correlation coefficient R² of 0.977.

Detection Principle:

ALP can conduct specific catalytic hydrolysis on a phosphate group. The hydrolysis principle of the ALP is as shown in FIG. 1. The ALP hydrolyzes 5-bromo-4-chloro-3-indolyl phosphate (BCIP) disodium salt without an SERS characteristic to obtain 5-bromo-4-chloro-3-indole (BCI), the produced BCI is rapidly oxidized to form a water-insoluble BCI oxidized dimer with a strong SERS characteristic, and the activity of the ALP is determined by establishing a correlation between different activities of the ALP and the intensity of the SERS characteristic peak of the product.

Theoretically, product BCI oxidized dimer has a characteristic Raman peak at a wave number of 600 cm⁻¹, which is verified by the experiment below.

200 μL of 1 mg/mL of a BCIP solution, 200 μL of 1 U/mL of an ALP solution and 200 μL of a DMSO solution are separately taken, after the BCIP (200 μL, 1 mg/mL) and the ALP (200 μL, 1 U/mL) react for 2 h, the obtained solution is placed in 4 sample vials on a machine, 200 μL of gold nanoparticle colloid is added and uniformly mixed, and an SERS signal of the obtained mixture was detected (at an excitation wavelength of 785 nm and an excitation frequency of 5 mW for 10 s and 30 Raman spectra are collected for each sample).

As shown in FIG. 2, the BCIP solution and the ALP solution have no SERS characteristics. The DMSO solution has no Raman peak near wave number 600 cm⁻¹, but has two obvious SERS characteristic peaks near wave numbers of 677 cm⁻¹ and 700 cm⁻¹, which represent a C—S—C symmetric stretching vibration peak and a C—S stretching vibration peak, respectively. The Raman peak at wave number of 677 cm⁻¹ is selected as an internal standard peak for quantitatively detecting the activities of extracellular enzymes. Wherein, the SERS signal of the BCI oxidized dimer formed by reacting BCIP and ALP for 2 h shows a relatively strong Raman peak at wave number 600 cm⁻¹. The Raman peak is caused by a C═C—CO—C plane vibration in a chemical structure of the product, that is a characteristic peak of the product.

### BRIEF DESCRIPTION OF THE DRAWINGS

FIG. 1 is a reaction equation of ALP hydrolyzing BCIP;

FIG. 2 shows surface-enhanced Raman spectroscopy (SERS) of an experimental solvent and a substrate;

FIG. 3a shows surface-enhanced Raman spectroscopy corresponding to different activities of ALP;

FIG. 3b is surface-enhanced Raman spectroscopy of selected wave numbers ranging from 400 cm⁻¹ to 800 cm⁻¹;

FIG. 4 shows fitting a standard curve by a linear equation; and

FIG. 5 is an SERS image after a seawater sample reacts with BCIP.

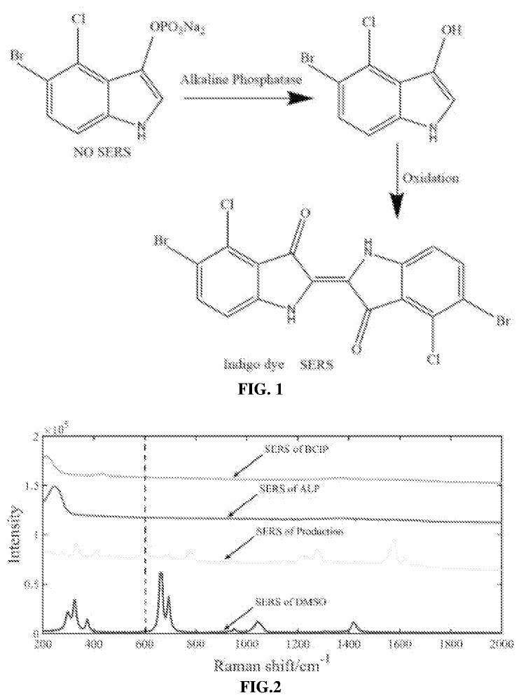

### DETAILED DESCRIPTION

The present disclosure will be further described in detail with reference to the accompanying drawings and specific examples.

### Example 1 Quantitative Model

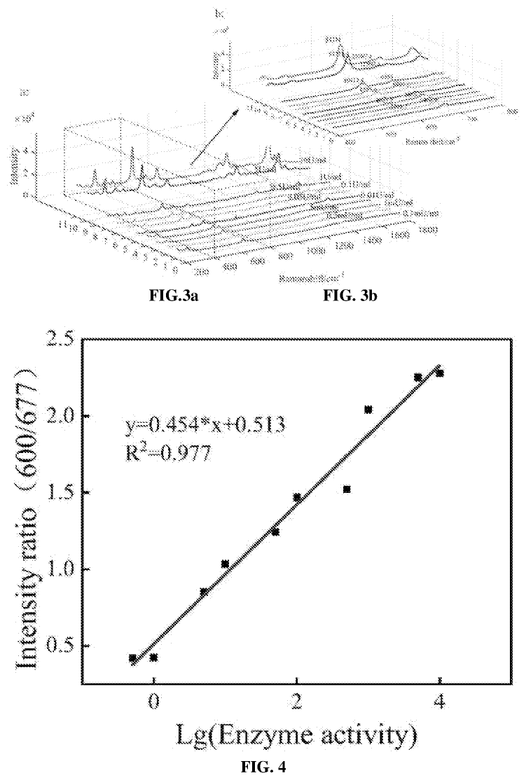

Eleven 900 μL of ALP solutions with different activities (10 U/mL, 5 U/mL, 1 U/mL, 0.5 U/mL, 0.1 U/mL, 50 mU/mL, 10 mU/mL, 5 mU/mL, 1 mU/mL, 0.5 mU/mL and 0.1 mU/mL) were separately mixed with 100 μL of 1 mg/mL of a BCIP solution for incubation for 2 h. After a DMSO solution with 20% volume was added, respective SERS signals were detected and obtained spectra were as shown in FIG. 3.

As can be seen from FIG. 3, there was no direct linear correlation between SERS characteristic peak intensity of a product BCI oxidized dimer and the concentration of the ALP. Therefore, it is difficult to directly use the intensity of the characteristic Raman peak to conduct quantitative analysis due to interference by factors of stability of laser power, uniformity of an enhancing reagent, background noise of a solvent, etc. Thus the characteristic Raman peak of the added DMSO solvent near wave number 677 cm⁻¹ was used as an internal standard peak. An internal standard method was used to establish a quantitative detection model to quantitatively detect the ALP. Table 1 shows intensity of SERS characteristic peaks of a substrate and an internal standard.

Table 3 Intensity of characteristic peaks of substrate and internal standard.

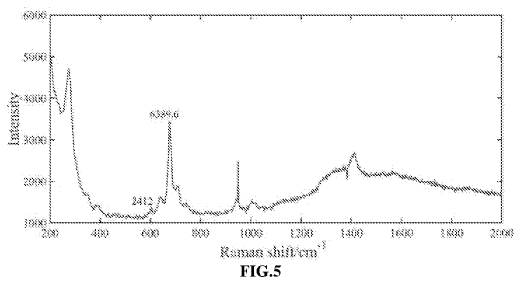

It can be seen from Table 1 that generally the intensity of the characteristic peak of the product (600 cm⁻¹) and the intensity of the internal standard peak (677 cm⁻¹) gradually decreased with decrease of the activity of the ALP, but there was no good functional relationship. RSD of a corresponding intensity ratio of each enzyme activity was less than 15%, indicating that the SERS data were highly reliable. A linear fitting was conducted on the concentration of the ALP and the SERS intensity ratio (600 cm⁻¹/677 cm⁻¹) by using a least squares method. The result is as shown in FIG. 4.

The DMSO was introduced as the internal standard, the characteristic peak at the wave number of 677 cm⁻¹ was used as the internal standard peak. Logarithm value of a total of 10 ALP activities of 10 U/mL, 5 U/mL, 1 U/mL, 0.5 U/mL, 0.1 U/mL, 50 mU/mL, 10 mU/mL, 5 mU/mL, 1 mU/mL and 0.5 mU/mL was taken as the x-coordinate, and the ratio of the product peak (600 cm⁻¹) to the internal standard peak (677 cm⁻¹) was taken as the y-coordinate. A standard equation was fitted: y=0.454*x+0.513 with a correlation coefficient R² of 0.977, indicating a strong linear correlation between ALP activity and the ratio of the intensity of the characteristic Raman peaks (600 cm⁻¹/677 cm⁻¹). The model was capable of quantitatively detecting ALP activity.

### Example 2 Verification Test of a Seawater Sample

A fresh seawater sample was collected from the East China Sea (30° 39′48″N, 122° 29′48″E) in December 2020. The sample was surface seawater and obtained by using a fishing boat. 900 μL of the fresh seawater sample was mixed with 100 μL of 1 mg/mL of a BCIP solution for incubation for 2 h, after a DMSO solution with 20% volume was added, a SERS signal was detected and an obtained spectrum was as shown in FIG. 5.

As shown in FIG. 5, there was an obvious Raman peak at 600 cm⁻¹, indicating that the technique successfully and qualitatively detected ALP in the seawater sample. A Raman intensity at wave number 677 cm⁻¹ reached 6389.6, which indicated a Raman peak caused by C—S—C symmetrical stretching vibration in the DMSO. The ratio of the two peaks (600 cm⁻¹/677 cm⁻¹) is 0.377, and the value was substituted into the above model, and ALP activity of the seawater sample was quantitatively detected, and the obtained ALP activity of the water sample was equivalent to activity of 0.5 mU/mL of ALP in Escherichia coli.

The technique for quantitatively detecting the activity of the ALP based on SERS by taking the BCIP as the substrate and the DMSO as the internal standard was provided. Results showed that ALP activity and the intensity ratio of the product characteristic peak to the internal standard peak (600 cm⁻¹/677 cm⁻¹) had a good linear correlation with a correlation coefficient of 0.977. The activity of ALP in the seawater sample was successfully and quantitatively detected by using the model, thus ALP activity of the seawater sample can be rapidly detected. Meanwhile, the technique could also be used for detecting the activity of other extracellular enzymes of microorganisms in seawater and lays a solid scientific foundation for in-situ detection of the activity of microbial extracellular enzymes in seawater.

The above description is only preferred embodiments of the present disclosure and not limited in the present disclosure. All equivalent modifications, equivalents, improvements, etc. made within the spirit and the principle of the present disclosure should be included in the scope of the present disclosure.

1. A technique for quantitatively detecting alkaline phosphatase in seawater based on surface-enhanced Raman spectroscopy, comprising the following steps:
a. preparing a variety of alkaline phosphatase samples with different activities in advance, separately mixing and incubating the alkaline phosphatase samples with a BCIP solution for a period of time, adding a DMSO solution as a standard solution;
b. dropping a plurality of standard solutions with different activities onto a surface-enhanced Raman scattering substrate separately, conducting an SERS detection separately, and drawing a standard curve representing relations between intensity ratios of obtained SERS signals of the standard solutions with different activities to obtained SERS signal of the DMSO, and the logarithm values of the activities of the standard solutions;
c. dropping a solution of a sample to be tested containing DMSO onto a surface-enhanced Raman scattering substrate to directly detect SERS signals of the sample to be tested and the DMSO; and
d. comparing the SERS signals obtained in step c with the standard curve to obtain the activity of the sample to be tested.

2. The technique for quantitatively detecting an alkaline phosphatase based on surface-enhanced Raman spectroscopy according to claim 1, wherein in step b and step c, the SERS signal of the DMSO is obtained by selecting a peak intensity of the DMSO at a Raman shift of 677 cm⁻¹.

3. The technique for quantitatively detecting an alkaline phosphatase based on surface-enhanced Raman spectroscopy according to claim 1, wherein in step b and step c, the SERS signal of the alkaline phosphatase is obtained by selecting a peak intensity of the alkaline phosphatase at a Raman shift of 600 cm⁻¹.

4. The technique for quantitatively detecting an alkaline phosphatase based on surface-enhanced Raman spectroscopy according to claim 1, wherein in step b, the standard curve has a fitted equation of y=0.454*x+0.513 with a correlation coefficient R₂ of 0.977.
